(19) United States
(12) Patent Application Publication
(10) Pub. No.: US 2024/0099831 A1
(43) Pub. Date: Mar. 28, 2024

(54) TREATMENT APPARATUS FOR A CATARACT TREATMENT OF AN EYE

(71) Applicant: Carl Zeiss Meditec AG, Jena (DE)

(72) Inventors: Jennifer-Magdalena Masch, Oberkochen (DE); Holger Matz, Oberkochen (DE); Simon Schroeder, Oberkochen (DE); Hristina Srbinoska, Oberkochen (DE); Delbert Andrews, Oberkochen (DE); Hessam Roodaki, Oberkochen (DE); Martin Hacker, Oberkochen (DE)

(21) Appl. No.: 18/373,258

(22) Filed: Sep. 26, 2023

(30) Foreign Application Priority Data
Sep. 26, 2022 (DE) 10 2022 124 677.9

(51) Int. Cl. A61F 2/16; A61B 3/103

(52) U.S. Cl. CPC A61F 2/1648; A61B 3/103; A61F 2002/1681

(57) ABSTRACT

A treatment apparatus for a cataract treatment of an eye includes a modular intraocular lens including a first part, which includes a haptic configured to contact a capsular bag of the eye in a region of the equator of the capsular bag and thereby to span the capsular bag, and a second part, which includes an optic body, and has a convergence state, in which the second part contacts the first part, and a spaced-apart state, in which the second part is arranged spaced apart from the first part, a measurement system configured to determine, based on a first measurement, a position of the first part in the eye and in the spaced-apart state of the modular intraocular lens, and a controller configured to determine, based on the position of the first part, a refractive power of the optic body which is to be inserted into the eye.

### CROSS REFERENCE TO RELATED APPLICATIONS

This application claims priority to German patent application DE 10 2022 124 677.9, filed Sep. 26, 2022, the entire content of which is incorporated herein by reference.

### TECHNICAL FIELD

The disclosure relates to a treatment apparatus for a cataract treatment of an eye.

### BACKGROUND

In a cataract treatment, a natural lens of an eye is replaced with an artificial intraocular lens. To this end, an incision with a length of approx. 2 mm is introduced into the eye. Via the incision, the natural lens is broken into small pieces, for example with phacoemulsification, and subsequently removed by suction. The intraocular lens is subsequently introduced into the eye via the incision. The eye can be measured prior to the cataract treatment, in order to determine the properties of the intraocular lens to be inserted, such as the refractive power thereof. Nevertheless, the position of the intraocular lens cannot be predicted exactly, such that in some cases the refractive power is not selected suitably. In addition, the eye changes as a result of the introduction of the incision into the eye and the removal by suction of the natural lens during the cataract treatment. As a result, a measurement carried out prior to the cataract treatment no longer corresponds to the actual conditions during the cataract treatment. This may lead to the insertion of an intraocular lens which does not have optimal properties for the eye. The non-optimal properties may lead to imaging aberrations of the intraocular lens on the retina of the eye.

### SUMMARY

It is therefore an object of the disclosure to provide a treatment apparatus for a cataract treatment of an eye, wherein imaging aberrations can be reduced with the treatment apparatus.

The treatment apparatus according to an aspect of the disclosure for a cataract treatment of an eye includes a modular intraocular lens, a measurement system and a computing unit. The modular intraocular lens includes a first part, which includes a haptic, and a second part, which includes an optic body. The haptic is configured to contact a capsular bag of the eye in a region of the equator of the capsular bag and, as a result, to span the capsular bag. In addition, the intraocular lens has a convergence state, in which the second part contacts the first part, and a spaced-apart state, in which the second part is arranged spaced apart from the first part. The measurement system is configured to determine, on the basis of a first measurement, a position of the first part in the eye and in the spaced-apart state of the modular intraocular lens. The computing unit is configured to determine, using the position of the first part, a refractive power of the optic body which is to be inserted into the eye. The optic body may, for example, be a lens.

During a cataract treatment, the natural lens is removed from the capsular bag of the eye. The first part can subsequently be inserted into the capsular bag, before the second part is inserted into the eye. Due to the fact that the first part includes the haptic, the first part spans the capsular bag. As a result, in this state the eye has approximately the shape it will after wound healing following the cataract treatment. By virtue of the computing unit being configured to use the first measurement, which is carried out on the eye in this state, as a basis to determine the refractive power of the optic body, the refractive power of the optic body can be selected with a high accuracy, before the optic body is inserted into the eye. As a result, imaging aberrations of the modular intraocular lens on the retina of the eye can be reduced.

To determine the refractive power of the optic body, a plurality of known methods and/or formulas are available. Exemplary formulas are: Holladay I, Holladay II, Haigis, ZCALC and Olcen C. Exemplary methods include regression models, ray-tracing simulations and/or machine learning methods.

The computing unit may, for example, include a processor. In particular, the computing unit may be formed by a computer.

The position of the first part may, for example, include a distance of the first part from the retina of the eye and/or from the cornea of the eye, in particular in the direction of the optical axis of the optic body and/or of the natural lens of the eye, said position having been found prior to the insertion of the first part in the eye. In addition or as an alternative, the position of the first part may include a distance from the fovea of the eye.

It is typical for the measurement system to be configured to determine, on the basis of the first measurement, the position of the first part, without needing to carry out a refraction measurement or a wavefront analysis for this. By way of example, this may be effected by virtue of the measurement system including an optical coherence tomography device.

It is typical for the measurement system to be configured to determine, on the basis of the first measurement, a first property of the eye, said first property being selected from the group: an axial length of the eye, a shape of the cornea, in particular a thickness of the cornea, a curvature of an outer side of the cornea, a curvature of an inner side of the cornea, a tomography of the cornea and/or a topography of the cornea, an orientation of the first part, wherein the computing unit is configured to determine, using the first property, the refractive power, a position of the optic body, which is to be inserted into the eye, in the eye and/or an orientation of the optic body, which is to be inserted into the eye, in the eye. Due to the fact that the first property has been measured on the eye, in which the first part of the modular intraocular lens is arranged in the spaced-apart state, the refractive power of the optic body can be determined even more precisely or the position and/or the orientation of the optic body can be determined with a high accuracy. It is also possible for a plurality of the first properties to be selected from the aforementioned list.

The modular intraocular lens is typically configured such that, in the convergence state, the optic body can be arranged in various positions relative to the first part. This makes it possible to correct imaging aberrations when the second part is arranged in the eye. By way of example, in the case that the optic body has a refractive power >0 diopters, a displacement of the second part toward the retina makes it possible to change the eye toward farsightedness and a displacement of the second part away from the retina makes it possible to change the eye toward nearsightedness.

The modular intraocular lens is typically configured such that, in the convergence state, the optic body can be arranged in various orientations relative to the first part. In particular, the optic body is mounted in the first part such that it can be pivoted or rotated about an optical axis of the optic body. As an alternative or in addition, the optic body is arranged on the first part in a tiltable manner.

It is typical for the measurement system to be configured to determine, on the basis of a second measurement of the eye with the modular intraocular lens in the convergence state and arranged in the eye, a second property of the eye, wherein the computing unit is configured to determine, using the second property, a change in the position of the optic body relative to the first part and/or a change in the orientation of the optic body relative to the first part. This makes it possible to even further reduce the imaging aberrations.

The first part typically includes a further optic body. The further optic body may be a light-transmissive element. The light-transmissive element can have any desired form, such as a lens, a disk or a plate. As an alternative to providing the further optic body, it is also conceivable for the first part to be optic body-free.

The second property is typically selected from the group: -an axial length of the eye, -a shape of the cornea, in particular a thickness of the cornea, a curvature of an outer side of the cornea, a curvature of an inner side of the cornea, a tomography of the cornea and/or a topography of the cornea, -an orientation of the first part, -an orientation of the second part, -an axial distance of the optic body from the further optic body of the modular intraocular lens, -a geometry of the optic body, in particular a curvature and/or a thickness of the optic body, -a geometry of the further optic body, in particular a curvature and/or a thickness of the further optic body. It is also possible for a plurality of the second properties to be selected from the aforementioned list.

The measurement system is typically configured to implement, on the basis of a preoperative measurement, a preoperative property of the eye with a natural lens arranged in the eye, wherein the computing unit is configured to determine, using the preoperative property, the refractive power of the optic body which is to be inserted into the eye.

It is typical for the preoperative property to be selected from the group: -an axial length of the eye, -a shape of the cornea, in particular a thickness of the cornea, a curvature of an outer side of the cornea, a curvature of an inner side of the cornea, a tomography of the cornea and/or a topography of the cornea. It is also conceivable for a plurality of the preoperative properties to be selected from the list. The computing unit is typically configured to determine, using the preoperative property, the refractive power, a position of the optic body, which is to be inserted into the eye, in the eye and/or an orientation of the optic body, which is to be inserted into the eye, in the eye.

The measurement system typically includes at least one measurement device which is selected from the group: an optical coherence tomography device, a surgical microscope, a confocal microscope, a wavefront analysis device (which is also known under the name aberrometer), such as a Shack-Hartmann sensor, an ultrasound analysis device, a magnetic resonance tomography device, a camera which includes a Scheimpflug adapter, a confocal or chromatic confocal distance measurement system.

It is typical for the refractive power to exhibit a refractive power of a sphere shape of the optic body, or wherein the refractive power exhibits a refractive power of a sphere shape of the optic body and a refractive power of a cylinder shape of the optic body and the computing unit is configured to determine an orientation of the cylinder shape in the eye, or wherein the refractive power varies over the optic body, in order to correct or control higher-order aberrations.

It is typical for the computing unit to be configured to correct the position of the first part by taking account of the presence of an operating liquid in the anterior chamber of the eye. In this case, it is for example possible to estimate a refractive index of the liquid arranged in the eye by virtue of a known structure of the first part being measured in the first measurement and the refractive index being varied until the structure in the first measurement approximates to the known structure. The operating liquid may, for example, be an ophthalmic viscoelastic device (OVD) and/or a physiological saline solution, which may be buffered.

The computing unit or controller is typically configured to determine, on the basis of a cornea measurement, a position and in particular a size of an incision via which the first part is to be introduced or has been introduced. It has been found that in the case that the incision has been introduced decentrally into the cornea measurement, the intraocular lens can also be arranged decentrally in the capsular bag. It is therefore possible for the computing unit to be configured to determine, using the position of the incision and in particular the size of the incision, the position of the optic body, which is to be inserted into the eye, relative to the first part. By way of example, it is possible to conduct a measurement series which is stored in the computing unit and in which the position of the incision and in particular the size of the incision and the position of the first part in the eye have been determined for various cataract treatments. By way of example, it is possible for the surgical microscope to be configured to carry out the cornea measurement. The computing unit may be configured to correct the cornea measurement in such a way that a curvature of the cornea is taken into account.

It is typical for the second part to include a further haptic or to include no further haptic.

### BRIEF DESCRIPTION OF THE DRAWINGS

The disclosure will now be described with reference to the drawings wherein:

FIG. 1 shows a schematic treatment apparatus,

FIG. 2 shows a plan view of a first exemplary embodiment of a modular intraocular lens,

FIG. 3 shows a plan view of a second exemplary embodiment of the modular intraocular lens,

FIG. 4 shows a plan view of a third exemplary embodiment of the modular intraocular lens,

FIG. 5 shows a plan view of a fourth exemplary embodiment of the modular intraocular lens,

FIG. 6 shows a longitudinal section through a fifth exemplary embodiment of the modular intraocular lens at a first point in time,

FIG. 7 shows the longitudinal section from FIG. 6 at a second point in time,

FIG. 8 shows a plan view of a first part of a sixth exemplary embodiment of the intraocular lens and a side view of a second part of the sixth exemplary embodiment,

FIG. 9 shows a longitudinal section through a seventh exemplary embodiment of the modular intraocular lens with a first arrangement of a second part of the seventh exemplary embodiment relative to a first part of the seventh exemplary embodiment,

FIG. 10 shows the longitudinal section from FIG. 9 with a second arrangement of the second part relative to the first part, and

FIG. 11 shows a plan view of an eighth exemplary embodiment of the modular intraocular lens.

### DESCRIPTION OF EXEMPLARY EMBODIMENTS

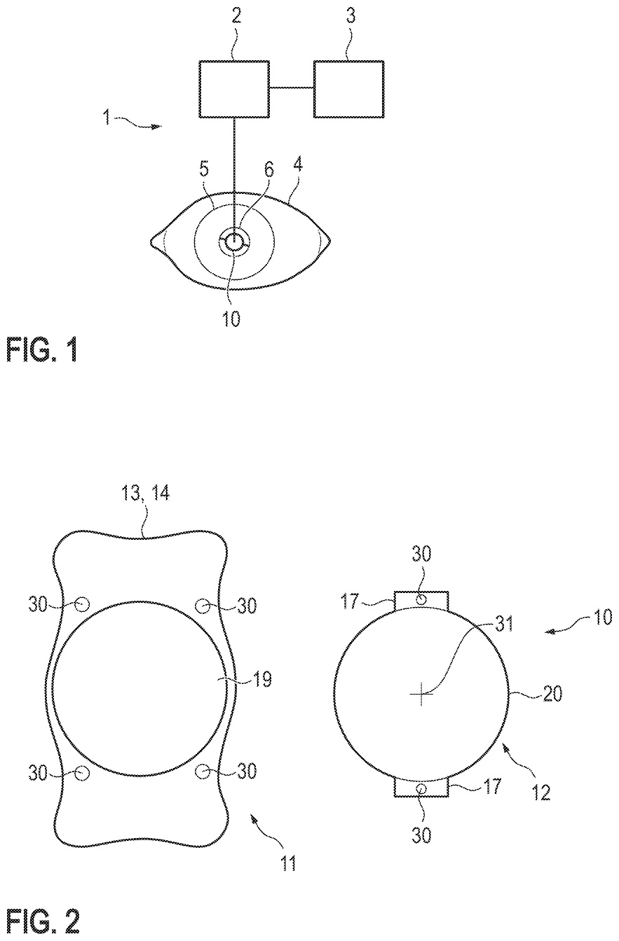

As is apparent from FIG. 1, a treatment apparatus 1 for a cataract treatment of an eye 4 includes a modular intraocular lens 10, a measurement system 2 and a computing unit 3. FIGS. 2 to 10 show that the modular intraocular lens 10 includes a first part 11, which includes a haptic 13, and a second part 12, which includes an optic body 20. The haptic 13 is configured to contact a capsular bag of the eye 4 in a region of the equator of the capsular bag and, as a result, to span the capsular bag. In particular, the haptic 13 may be configured to not contact the capsular bag in a region of an opening made by capsulorhexis on a front side of a capsular bag, i.e., the haptic 13 is configured to leave the opening formed by capsulorhexis free. In other words: the haptic 13 is configured to not contact the capsular bag on the outer side thereof. The capsulorhexis is an incision introduced into the capsular bag on the front side thereof during the cataract treatment. In addition, the modular intraocular lens 10 has a convergence state, in which the second part 12 contacts the first part 11 (cf. FIGS. 6, 7, 9, and 10), and a spaced-apart state, in which the second part 12 is arranged spaced apart from the first part 11 (cf. FIGS. 2 to 5, 8, and 11). The measurement system 2 is configured to determine, on the basis of a first measurement, a position of the first part 11 in the eye 4 and in the spaced-apart state of the modular intraocular lens 10. The computing unit 3 is configured to determine, using the position of the first part 11, a refractive power of the optic body 20 which is to be inserted into the eye 4. FIG. 1 shows the eye 4 with an eyeball 5 and the modular intraocular lens 10, which is visible through the pupil 6 of the eye 4.

The measurement system 2 may be configured to measure the eye 4 from the cornea of the eye 4 to the retina of the eye 4. It is also conceivable for the measurement system 2 to be configured to measure an anterior region of the eye 4, in particular in a region from the cornea of the eye 4 into the interior of the capsular bag of the eye 4. The measurement system 2 may include at least one measurement device which is selected from the group: an optical coherence tomography device, a surgical microscope, a confocal microscope, a wavefront analysis device, such as a Shack-Hartmann sensor, an ultrasound analysis device, a magnetic resonance tomography device, a camera which includes a Scheimpflug adapter, a confocal or chromatic confocal distance measurement system.

FIGS. 6, 7, 9, and 10 illustrate a coordinate system with an x axis, a y axis and a z axis. Here, the z axis is oriented in the direction of the optical axis of the natural lens. The x axis and the y axis are oriented perpendicularly with respect to the z axis and perpendicularly with respect to one another.

As is apparent from FIGS. 2 to 11, the modular intraocular lens 10 may be configured such that, in the convergence state, the optic body 20 can be arranged in various positions and/or in various orientations relative to the first part 11.

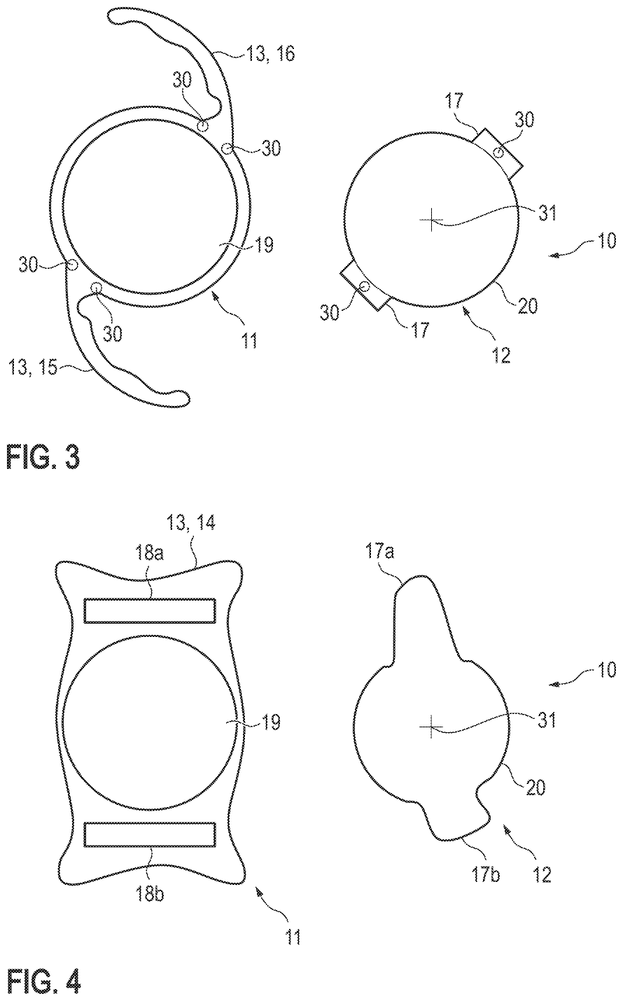

FIGS. 2 and 3 show that, in the convergence state, the optic body 20 may be mounted such that the optic body 20 can be pivoted or rotated about the z axis. To this end, the first part 11 may include a cutout 19 in which the optic body 20 is arranged in the convergence state. The second part 12 may include a protrusion 17 which is fastened to the optic body 20 and protrudes from the optic body 20 in a radial direction with respect to an optical axis 31 of the optic body 20. During a cataract treatment, a surgeon can grip the protrusion 17 with a tool and thus pivot the optic body 20 about the z axis. It is also conceivable for the second part 12 to include a plurality of the protrusions 17. It is conceivable for the haptic 13 to be a plate haptic 14 (cf. FIG. 2), or for the haptic 13 to be in the shape of a C or in the shape of a J (cf. FIG. 3). FIG. 3 shows that the haptic 13 may include a first haptic arm 15 and a second haptic arm 16, which may in particular be arranged spaced apart from one another.

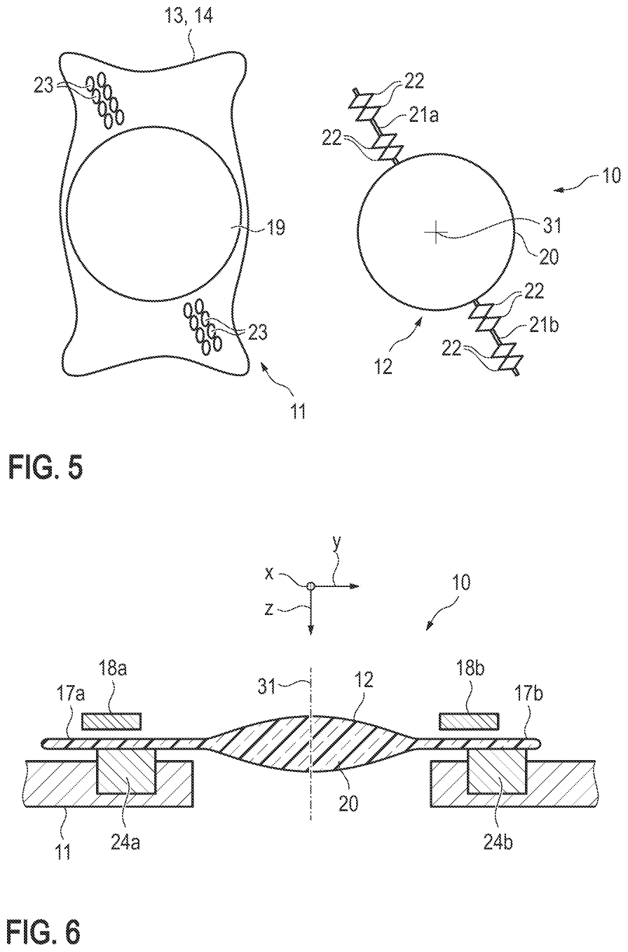

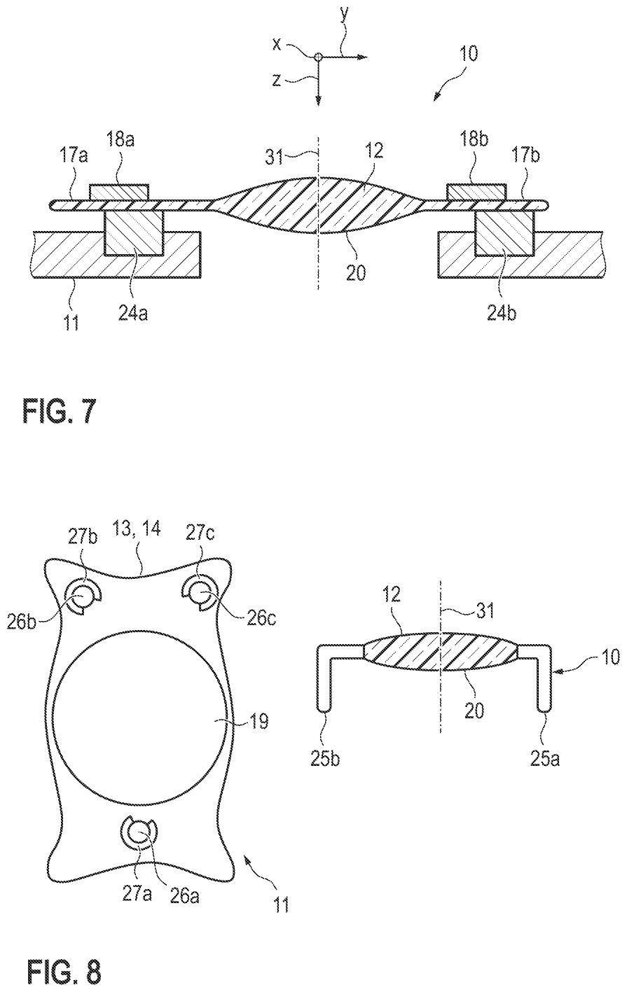

FIGS. 4 and 5 illustrate two exemplary embodiments in which the optic body 20 can be arranged in various positions in the x direction and/or y direction. In the exemplary embodiment according to FIG. 4, the first part 11 includes a first tab 18a and the second part 12 includes a first protrusion 17a which is fastened to the optic body 20 and protrudes outward in a radial direction with respect to an optical axis 31 of the optic body 20. The first part 11 includes a second tab 18b and the second part 12 includes a second protrusion 17b which is fastened to the optic body 20 and protrudes outward in a radial direction with respect to an optical axis 31 of the optic body 20. In the convergence state, the first protrusion 17a is arranged in the first tab 18a and the second protrusion 17b is arranged in the second tab 18b (cf. FIG. 7). The protrusions 17a, 17b are arranged in the respective tabs 18a, 18b with play, such that the optic body 20 can be displaced in the x direction and/or in the y direction relative to the first part 11. It is conceivable for the second protrusion 17b to be configured in a non-symmetrical manner with respect to the optical axis 31 in relation to the first protrusion 17a. As a result, and by arranging the first protrusion 17a either in the first tab 18a or in the second tab 18b, a displacement of the optic body 20 relative to the first part 11 along a long distance in the x direction and/or the y direction is possible. FIG. 4 shows that the haptic 13 may be a plate haptic 14. In the exemplary embodiment according to FIG. 5, the first part 11 may include a plurality of anchoring cutouts 23 which can at least partially be arranged at different distances from the optic body 20. In particular, the haptic 13 may be a plate haptic 14 and the anchoring cutouts 23 may be arranged in the plate haptic 14. The second part 12 may include a first anchoring 21a which includes at least one anchoring protrusion 22 which engages into one of the anchoring cutouts 23 in the convergence state. By virtue of the anchoring protrusion 22 engaging into different ones of the anchoring cutouts 23, which are arranged at the different distances from the optic body 20, the optic body 20 can be arranged in various positions in the x direction and/or the y direction relative to the first part 11. FIG. 5 also shows that the second part 12 may include a second anchoring 21b which may include at least one anchoring protrusion 22. The anchoring protrusion 22 may likewise engage into one of the anchoring cutouts 23 in the convergence state. As a result, the second part 12 is attached to the first part 11 more firmly than when only one anchoring protrusion 22 engages into one of the anchoring cutouts 23.

FIGS. 6 and 7 show that the second part 12 may be fastened to the first part for example with friction. FIG. 6 shows a first point in time at which the second part 12 is not yet fastened to the first part 11, and FIG. 7 shows a second point in time at which the second part 12 is fastened to the first part 11. FIGS. 6 and 7 show that the first part 11 may include a first clamping apparatus 24a and optionally a second clamping apparatus 24b, which can each change their shape by way of an external stimulus, such as heating and/or application of electromagnetic radiation, and thus fasten the second part 12 to the first part 11. To this end, it is for example possible for the first clamping apparatus 24a and optionally the second clamping apparatus 24b to include a shape memory material, such as nitinol.

FIGS. 8 to 10 show two exemplary embodiments for the modular intraocular lens 10 in which the position of the optic body 20 can be changed in the z direction relative to the first part 11 and the orientation of the optic body 20 can be changed relative to the first part 11 such that tilting with respect to the x direction and/or tilting with respect to the y direction at different angles is possible. FIG. 8 shows that the second part 12 may include a first pin 25a and a second pin 25b which project in the direction of the optical axis 31 beyond the optic body 20. The first part 11 includes a first pin through-hole 26a and a second pin through-hole 26b. In the convergence state, the first pin 25a engages into the first pin through-hole 26a and the second pin 25b engages into the second pin through-hole 26b. By virtue of the first pin 25a and the second pin 25b being displaced by a distance of equal length relative to the associated pin through-hole 26a, 26b, the position of the optic body 20 can be changed in the z direction. By virtue of the first pin 25a and the second pin 25b being displaced by a distance of different length relative to the associated pin through-hole 26a, 26b, the optic body 20 can be inclined in relation to the first part. In addition, the optic body 20 may include a third pin (concealed by the second pin 25b in FIG. 8) and the first part 11 may include a third pin through-hole 26c into which the third pin engages in the convergence state. It is conceivable for the first part 11 to include a first clip 27a, which clamps the first pin 25a in the convergence state, and a second clip 27b, which clamps the second pin 25b in the convergence state. In particular, the first part includes a third clip 27c, which clamps the third pin in the convergence state. It is conceivable for the haptic 13 to include a plate haptic 14 into which the first pin through-hole 26a and the second pin through-hole 26b and in particular the third pin through-hole 26c are introduced.

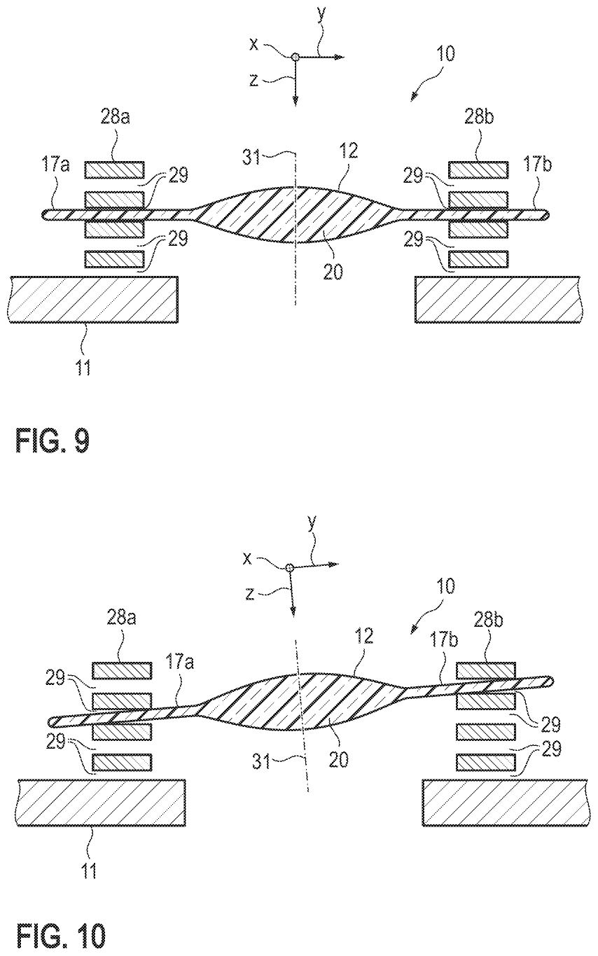

In the exemplary embodiment according to FIG. 10, the first part 11 includes a first rack 28a and a second rack 28b. The first rack 28a includes a plurality of compartments 29 which are arranged next to one another in the direction of the optical axis 31, and the second rack 28b includes a plurality of compartments 29 which are arranged next to one another in the direction of the optical axis 31. The second part 12 includes a first protrusion 17a and a second protrusion 17b which are fastened to the optic body 20 and protrude from the optic body 20 in a radial direction with respect to the optical axis 31 and are configured to engage into a respective one of the compartments 29. It is for example possible for the first protrusion 17a and the second protrusion 17b to engage into compartments 29 which are arranged in an equal position in the z direction (cf. FIG. 9) or which are arranged in different positions in the z direction (cf. FIG. 10).

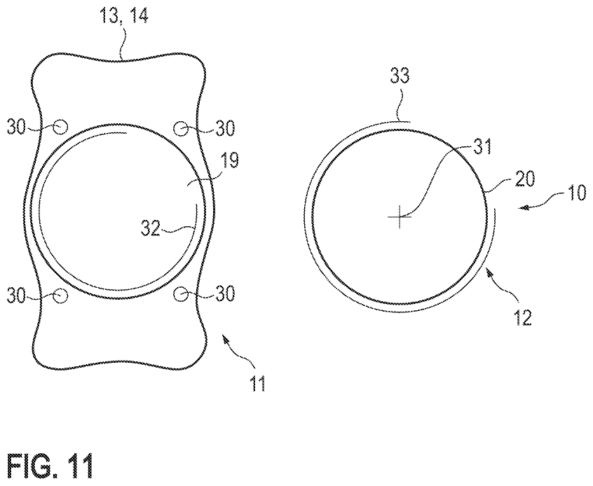

In the exemplary embodiment according to FIG. 11, the first part 11 includes an internal thread 32, and the second part 12 includes an external thread 33 which is configured to be screwed into the internal thread 32. Depending on how far the external thread 33 is screwed into the internal thread 32, the position of the optic body 20 can be adjusted in the z direction relative to the first part 11.

FIGS. 2, 3, and 11 show that the first part 11 may include a marking 30 or a plurality of markings 30 which can be detected by the measurement system 2. This applies in particular when the measurement system 2 includes the optical coherence tomography device. The marking 30 may, for example, include a cutout, in particular an engraving, and/or a protrusion. The marking 30 may also include a material different than the material that surrounds the marking 30. The marking 30 may, for example, be arranged such that the marking 30 is arranged tilted in relation to the optical axis 31.

It is conceivable for the second part 12 to include a further haptic or to include no further haptic.

It is understood that the foregoing description is that of the exemplary embodiments of the disclosure and that various changes and modifications may be made thereto without departing from the spirit and scope of the disclosure as defined in the appended claims.

### LIST OF REFERENCE NUMERALS

1 Treatment apparatus
2 Measurement system
3 Computing unit, controller
4 Eye
5 Eyeball
6 Pupil
10 Modular intraocular lens
11 First part
12 Second part
13 Haptic
14 Plate haptic
15 First haptic arm
16 Second haptic arm
17 Protrusion
17a First protrusion
17b Second protrusion
18a First tab
18b Second tab
19 Cutout
20 Optic body
21a First anchoring
21b Second anchoring
22 Anchoring protrusion
23 Anchoring cutout
24a First clamping apparatus
24b Second clamping apparatus
25a First pin
25b Second pin
26a First pin through-hole
26b Second pin through-hole
26c Third pin through-hole
27a First clip
27b Second clip
27c Third clip
28a First rack
28b Second rack
29 Compartment
30 Marking
31 Optical axis
32 Internal thread
33 External thread

What is claimed is:

1. A treatment apparatus for a cataract treatment of an eye, the treatment apparatus comprising:
a modular intraocular lens including a first part and a second part, the first part including a haptic configured to contact a capsular bag of the eye in a region of the equator of the capsular bag and thereby to span the capsular bag, the second part including an optic body, and having a convergence state, in which the second part contacts the first part, and a spaced-apart state, in which the second part is arranged spaced apart from the first part,
a measurement system configured to determine, based on a first measurement, a position of the first part in the eye and in the spaced-apart state of the modular intraocular lens, and
a controller configured to determine, with the position of the first part, a refractive power of the optic body which is to be inserted into the eye.

2. The treatment apparatus as claimed in claim 1, wherein the measurement system is further configured to determine, based on the first measurement, a first property of the eye, said first property being selected from the group consisting of:
an axial length of the eye,
a shape of the cornea, a thickness of the cornea, a curvature of an outer side of the cornea, a curvature of an inner side of the cornea, a tomography of the cornea and/or a topography of the cornea, and
an orientation of the first part,
wherein the controller is configured to determine, based on the first property, the refractive power, a position of the optic body, which is to be inserted into the eye, in the eye and/or an orientation of the optic body, which is to be inserted into the eye, in the eye.

3. The treatment apparatus as claimed in claim 1, wherein the modular intraocular lens is configured such that, in the convergence state, the optic body can be arranged in various positions and/or in various orientations relative to the first part.

4. The treatment apparatus as claimed in claim 3, wherein the measurement system is configured to determine, based on a second measurement of the eye with the modular intraocular lens in the convergence state and arranged in the eye, a second property of the eye, and
wherein the controller is configured to determine, based on the second property, a change in the position of the optic body relative to the first part and/or a change in the orientation of the optic body relative to the first part.

5. The treatment apparatus as claimed in claim 4, wherein the second property is selected from the group consisting of:
an axial length of the eye,
a shape of the cornea, a thickness of the cornea, a curvature of an outer side of the cornea, a curvature of an inner side of the cornea, a tomography of the cornea, and/or a topography of the cornea,
an orientation of the first part,
an orientation of the second part,
an axial distance of the optic body from a further optic body of the modular intraocular lens,
a geometry of the optic body, a curvature and/or a thickness of the optic body, and
a geometry of the further optic body, a curvature and/or a thickness of the further optic body.

6. The treatment apparatus as claimed in claim 1, wherein the measurement system is configured to implement, based on a preoperative measurement, a preoperative property of the eye with a natural lens arranged in the eye, and
wherein the controller is further configured to determine, based on the preoperative property, the refractive power of the optic body which is to be inserted into the eye.

7. The treatment apparatus as claimed in claim 6, wherein the preoperative property is selected from the group consisting of:
an axial length of the eye, and
a shape of the cornea, a thickness of the cornea, a curvature of an outer side of the cornea, a curvature of an inner side of the cornea, a tomography of the cornea, and/or a topography of the cornea.

8. The treatment apparatus as claimed in 1, wherein the controller is configured to determine, based on the preoperative property, the refractive power, a position of the optic body, which is to be inserted into the eye, in the eye and/or an orientation of the optic body, which is to be inserted into the eye, in the eye.

9. The treatment apparatus as claimed in claim 1, wherein the measurement system includes at least one measurement device selected from the group consisting of:
an optical coherence tomography device,
a surgical microscope,
a confocal microscope,
a wavefront analysis device, such as a Shack-Hartmann sensor,
an ultrasound analysis device,
a magnetic resonance tomography device,
a camera which includes a Scheimpflug adapter, and
a confocal or chromatic confocal distance measurement system.

10. The treatment apparatus as claimed claim 1, wherein the refractive power exhibits a refractive power of a sphere shape of the optic body, or
wherein the refractive power exhibits a refractive power of a sphere shape of the optic body and a refractive power of a cylinder shape of the optic body and the controller is configured to determine an orientation of the cylinder shape in the eye, or
wherein the refractive power varies over the optic body, to correct or control higher-order aberrations.
